(19) United States
(12) Patent Application Publication
(10) Pub. No.: US 2021/0353882 A1
(43) Pub. Date: Nov. 18, 2021

(54) Coating a Respiratory System

(71) Applicant: Yigal Cohen Harel, Weston, FL (US)

(72) Inventor: Yigal Cohen Harel, Weston, FL (US)

(21) Appl. No.: 16/877,047

(22) Filed: May 18, 2020

(51) Int. Cl. A61M 15/00; A61M 11/00; A61M 15/06; A61K 45/06

(52) U.S. Cl. CPC A61M 15/0065; A61K 45/06; A61M 15/06; A61M 11/00

(57) ABSTRACT

Methods and devices for coating a respiratory system and thereby protecting from or killing viral, bacterial, or viral and bacterial infections are disclosed. An active compound that is a virucide, a bactericide, or both, is vaporized by a vaporizing device and inhaled into the respiratory system where the active compound coats the respiratory system.

### TECHNICAL FIELD

The inventions described herein relate generally to coating respiratory systems.

### BACKGROUND

Vaporizing liquids to aerosols, such as in vaping devices, is used for delivery of chemicals to users. This is commonly done to deliver nicotine or other chemicals that produce pleasant effects in the user.

### SUMMARY

In a first aspect, the disclosure provides a method for protecting a respiratory system. A feed liquid, containing an active compound, is vaporized by a vaporizing device to form a feed vapor. The feed vapor is inhaled into the respiratory system. The respiratory system is coated with the active compound. The active compound is a virucide, a bactericide, or both. Coating the respiratory system with the active compound protects the respiratory system.

In a second aspect, the disclosure provides a method for fighting or preventing a viral, bacterial, or viral and bacterial infection of the respiratory system. A feed liquid, containing an active compound, is vaporized by a vaporizing device to form a feed vapor. The feed vapor is inhaled into the respiratory system. The respiratory system is coated with the active compound. The active compound is a virucide, a bactericide, or both. Coating the respiratory system kills the viral, bacterial, or viral and bacterial infection.

In a third aspect, the disclosure provides a feed liquid for use in a vaporizing device. The feed liquid contains an active compound that is a virucide, a bactericide, or both. The feed liquid is vaporized by the vaporizing device to form a feed vapor and the feed vapor coats a respiratory system with the active compound.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a cross-sectional view of a human respiratory system and vape.

FIG. 2 is a cross-sectional view of a human respiratory system and metered-dose inhaler.

FIG. 3 is a block diagram showing a method for protecting a respiratory system.

FIG. 4 is a block diagram showing a method for fighting or preventing a viral, bacterial, or viral and bacterial infection of the respiratory system.

FIG. 5 is a cross-sectional view of a human respiratory system and nebulizer.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “respiratory system” is meant to refer to all parts of the human respiratory system, including the mouth, nose, sinuses, throat, trachea, lungs, and any other body part that contacts the same.

As used herein, “e-liquid” is meant to refer to e-cig liquid, juice, vapor juice, smoke juice, vaping fluid, vaping juice, e-juice, e-fluid, vape oil, or other liquid that is vaporized by a vape and delivered to the respiratory system.

As used herein, “vape” is meant to refer to any e-cigarette, electronic cigarette, vaporizer, vape pens, hookah pens, e-pipes, and any other device used to vaporize an e-liquid for delivery to a user's respiratory system.

As used herein, a “vaporizing device” is meant to refer to vapes, nebulizers, metered-dose inhalers, and any other device that vaporizes a liquid.

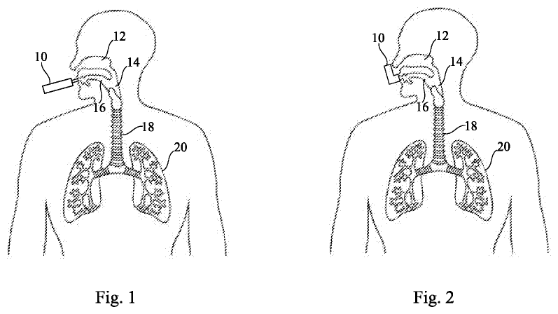

Now referring to FIG. 1, FIG. 1 is a cross-sectional view of a human respiratory system and vape that may be used in one embodiment of the present invention. A feed liquid consisting of an active compound is contained within a vape 10. The active compound is a virucide, a bactericide, or both. The vape 10 vaporizes the active compound, forming a feed vapor that is inhaled by the user. The feed vapor coats the respiratory system, including the mouth 16, the throat 14, the sinuses and nose 12, the trachea 18, and the lungs 20. Coating these introduces the active component onto the surfaces. In one embodiment, the active compound protects the respiratory system from viral infections, bacterial infections, or both. In some embodiments, the active compound blocks the human ACE2 receptor, blocking the virus from entering the respiratory systems' cells. In another embodiment, the active compound kills viral infections, bacterial infections, or both.

In some embodiments, a plurality of active compounds are included in the vape 10. Active compounds include, but are not limited to, alcohols, lemon oil, lavender oil, peppermint oil, thyme oil, eucalyptus oil, tea tree oil, chamomile oil, clove oil, cinnamon oil, rosemary oil, bergamot oil, wild carrot oil, Melissa oil, oregano oil, star anise oil, ginger oil, manuka oil, vanilla oil, preserving agents, antiseptic agents, disinfectants, benzalkonium chloride, benzoic acid, chlorocresol, chlorhexidine, benzethonium chloride, and combinations thereof. In a preferred embodiment, the active compound consists of between 0.5 wt % and 5 wt % of the feed liquid.

FIG. 2 is a cross-sectional view of a human respiratory system and metered-dose inhaler that may be used in one embodiment of the present invention. A feed liquid consisting of an active compound is contained within a metered-dose inhaler 10. The active compound is a virucide, a bactericide, or both. The inhaler 10 vaporizes the active compound, forming a feed vapor that is inhaled by the user. The feed vapor coats the respiratory system, including the mouth 16, the throat 14, the sinuses and nose 12, the trachea 18, and the lungs 20. Coating these introduces the active component onto the surfaces. In one embodiment, the active compound protects the respiratory system from viral infections, bacterial infections, or both. In another embodiment, the active compound kills viral infections, bacterial infections, or both.

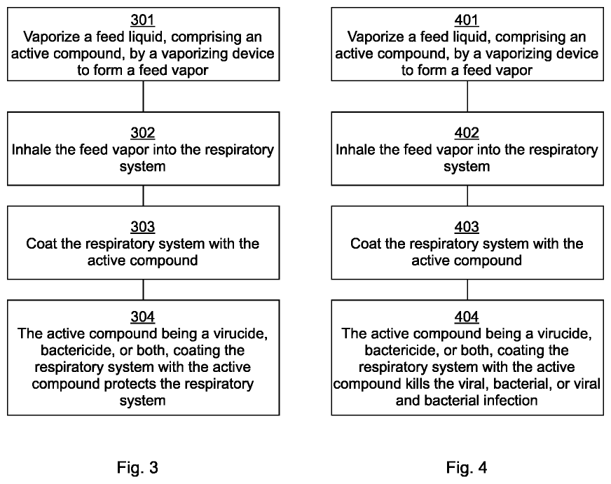

FIG. 3 is a block diagram showing a method for protecting a respiratory system that may be used in one embodiment of the present invention. At 301, a feed liquid, consisting of an active compound, is vaporized by a vaporizing device to form a feed vapor. At 302, the feed vapor is inhaled into the respiratory system. At 303, the active compound coats the respiratory system. At 304, the active compound, which is a virucide, bactericide, or both, coats the respiratory system with the active compound and thereby protects the respiratory system from viral or bacterial infection.

FIG. 4 is a block diagram showing a method for fighting or preventing a viral, bacterial, or viral and bacterial infection of the respiratory system that may be used in one embodiment of the present invention. At 401, a feed liquid, consisting of an active compound, is vaporized by a vaporizing device to form a feed vapor. At 402, the feed vapor is inhaled into the respiratory system. At 403, the active compound coats the respiratory system. At 404, the active compound, which is a virucide, bactericide, or both, coats the respiratory system with the active compound and kills the viral, bacterial, or viral and bacterial infection.

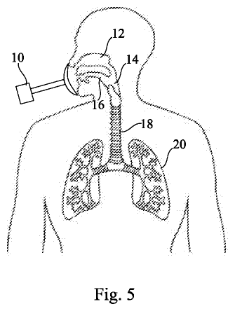

FIG. 5 is a cross-sectional view of a human respiratory system and nebulizer that may be used in one embodiment of the present invention. A feed liquid consisting of an active compound is contained within a nebulizer 10. The active compound is a virucide, a bactericide, or both. The nebulizer 10 vaporizes the active compound, forming a feed vapor that is inhaled by the user. The feed vapor coats the respiratory system, including the mouth 16, the throat 14, the sinuses and nose 12, the trachea 18, and the lungs 20. Coating these introduces the active component onto the surfaces. In one embodiment, the active compound protects the respiratory system from viral infections, bacterial infections, or both. In another embodiment, the active compound kills viral infections, bacterial infections, or both.

The invention has been described with reference to various specific and preferred embodiments and techniques. Nevertheless, it is understood that many variations and modifications may be made while remaining within the spirit and scope of the invention.

What is claimed is:

1. A method for protecting a respiratory system comprising:
vaporizing a feed liquid, comprising an active compound, by a vaporizing device to form a feed vapor;
inhaling the feed vapor into the respiratory system;
coating the respiratory system with the active compound;
wherein the active compound is a virucide, a bactericide, or both, and wherein coating the respiratory system with the active compound protects the respiratory system.

2. The invention of claim 1, wherein the active compound blocks the human ACE2 receptor.

3. The invention of claim 1, wherein the feed liquid is an e-liquid and the vaporizing device is a vape device.

4. The invention of claim 1, wherein the vaporizing device is a nebulizer or a metered-dose inhaler.

5. The invention of claim 1, wherein the active compound comprises from 0.5 wt % to 5 wt % of the feed liquid.

6. The invention of claim 1, wherein the active compound is a compound selected from the group consisting of alcohols, lemon oil, lavender oil, peppermint oil, thyme oil, eucalyptus oil, tea tree oil, chamomile oil, clove oil, cinnamon oil, rosemary oil, bergamot oil, wild carrot oil, Melissa oil, oregano oil, star anise oil, ginger oil, manuka oil, vanilla oil, preserving agents, antiseptic agents, disinfectants, benzalkonium chloride, benzoic acid, chlorocresol, chlorhexidine, benzethonium chloride, and combinations thereof.

7. The invention of claim 1, wherein the feed liquid comprises a plurality of active compounds.

8. A method for fighting or preventing a viral, bacterial, or viral and bacterial infection of the respiratory system comprising:
vaporizing a feed liquid, comprising an active compound, by a vaporizing device to form a feed vapor;
inhaling the feed vapor into the respiratory system;
coating the respiratory system with the active compound;
wherein the active compound is a virucide, a bactericide, or both, and wherein coating the respiratory system kills the viral, bacterial, or viral and bacterial infection.

9. The invention of claim 8, wherein the active compound blocks the human ACE2 receptor.

10. The invention of claim 8, wherein the feed liquid is an e-liquid and the vaporizing device is a vape device.

11. The invention of claim 8, wherein the vaporizing device is a nebulizer or a metered-dose inhaler.

12. The invention of claim 8, wherein the active compound comprises from 0.5 wt % to 5 wt % of the feed liquid.

13. The invention of claim 8, wherein the active compound is a compound selected from the group consisting of alcohols, lemon oil, lavender oil, peppermint oil, thyme oil, eucalyptus oil, tea tree oil, chamomile oil, clove oil, cinnamon oil, rosemary oil, bergamot oil, wild carrot oil, Melissa oil, oregano oil, star anise oil, ginger oil, manuka oil, vanilla oil, preserving agents, antiseptic agents, disinfectants, benzalkonium chloride, benzoic acid, chlorocresol, chlorhexidine, benzethonium chloride, and combinations thereof.

14. The invention of claim 8, wherein the feed liquid comprises a plurality of active compounds.

15. A feed liquid for use in a vaporizing device, the feed liquid comprising:
an active compound that is a virucide, a bactericide, or both; and
wherein the feed liquid is vaporized by the vaporizing device to form a feed vapor and the feed vapor coats a respiratory system with the active compound.

16. The invention of claim 15, wherein the active compound blocks the human ACE2 receptor.

17. The invention of claim 15, wherein the feed liquid is an e-liquid and the vaporizing device is a vape device.

18. The invention of claim 15, wherein the vaporizing device is a nebulizer or a metered-dose inhaler.

19. The invention of claim 15, wherein the active compound comprises from 0.5 wt % to 5 wt % of the feed liquid.

20. The invention of claim 15, wherein the active compound is a compound selected from the group consisting of alcohols, lemon oil, lavender oil, peppermint oil, thyme oil, eucalyptus oil, tea tree oil, chamomile oil, clove oil, cinnamon oil, rosemary oil, bergamot oil, wild carrot oil, Melissa oil, oregano oil, star anise oil, ginger oil, manuka oil, vanilla oil, preserving agents, antiseptic agents, disinfectants, benzalkonium chloride, benzoic acid, chlorocresol, chlorhexidine, benzethonium chloride, and combinations thereof.
